(19) United States
(12) Patent Application Publication
(10) Pub. No.: US 2019/0233793 A1
(43) Pub. Date: Aug. 1, 2019

(54) BIOCOMPATIBLE MATRICES FOR THE TRANSFER OF BIOLOGICAL MOLECULES

(71) Applicant: UNIVERSITY OF LEEDS, Leeds (GB)

(72) Inventor: Georg Feichtinger, Leeds (GB)

(21) Appl. No.: 16/319,000

(22) PCT Filed: Jul. 20, 2017
(86) PCT No.: PCT/GB2017/052139

§ 371 Date: Jan. 18, 2019

(30) Foreign Application Priority Data
Jul. 21, 2016 (GB) 1612625.2

(51) Int. Cl. C12N 5/00; C25D 13/04; A61K 9/00; A61P 17/02; A61K 9/51; C12N 5/0793; C12N 5/077; C12N 5/0775; A61K 35/36; A61K 35/28; A61K 35/32; A61K 35/30; A61K 39/00

(52) U.S. Cl. CPC C12N 5/0062; C12N 2533/90; A61K 9/0053; A61P 17/02; A61K 9/51; C12N 5/0619; C12N 5/0654; C12N 5/0663; A61K 35/36; A61K 35/28; A61K 35/32; A61K 35/30; A61K 39/00; C12N 2501/155; C12N 2501/15; C12N 2501/20; C25D 13/04

(57) ABSTRACT

There is provided a biocompatible material for delivering a biological molecule to target location, the material comprising: —a hydrogel matrix material, —a divalent cation-phosphate nanoparticle (in particular Calcium Phosphate), —and a biological molecule (in particular a nucleic acid) complexed with the nanoparticle; wherein the nanoparticle is embedded within the hydrogel matrix material. The biocompatible material, particularly when in a 3D form, can be used in the treatment of various diseases. A preferred method of embedding the nanoparticles and biological molecules in the matrix is by electrophoretic transfer.

### FIELD OF THE INVENTION

Certain aspects of the present invention relate to materials which may have utility as scaffold material for in vivo use. Also encompassed by certain aspects of the present invention are methods of producing such materials as well as methods of treating various disorders using such materials.

### BACKGROUND TO THE INVENTION

Non-viral gene therapeutics are considered a promising technology for tissue regenerative therapies and a plethora of other applications such as for up- and down-regulation of endogenous gene expression, vaccination and genome editing. Furthermore, non-viral methods using nucleic acids have an excellent safety profile compared to viral vectors. The use of non-viral genetic templates that target endogenous cells to translate the encoded information to actual cues in a controlled 3D environment in vivo have the potential to revolutionise current treatment approaches in tissue regeneration. By enabling the effective non-viral gene transfer to cells in vivo, such therapies can deliver a differentiation stimulus more precisely, at lower doses and in a sustained manner and with higher bioactivity compared to the administration of recombinant growth factors as transfected endogenous cells produce the growth factor locally. In an ideal scenario in the future, such cost-effective and targeted approaches to transient genetic manipulation in vivo could substitute for the expensive and cumbersome cell and growth factor therapies currently in use. The combination of transfection-grade plasmid DNA with a delivery agent and a biomaterial in a gene-activated matrix design (GAM) simultaneously supporting tissue regeneration and delivering therapeutic DNA to endogenous cells has therefore been the focus of intense research in the past.

A major limitation of current GAM systems, however, is their limited efficacy in gene delivery and lack of spatial control of transgene delivery. These are important attributes for clinical translation as the regeneration of complex tissues and tissue interfaces (for example, for regeneration of osteochondral defects within joints), in order to deliver multiple, spatially-restricted cues in order to orchestrate complex tissue formation.

Currently, biomaerials designed to address regeneration of complex tissue architectures are either fabricated as biomatrices with a gradient in mineralisation and/or by combination of different matrix materials in order to provide a scaffold material for endogenous regeneration. Many of these approaches require the additional application of specific adult precursor or stem cells in order to unlock their potential for tissue formation and do not deliver an active differentiation cue for regeneration. While all these solutions promise to benefit the regeneration of endogenous tissues, none have so far provided true functional regeneration of complex tissues and there are significant drawbacks associated with the cost of some of the materials, the availability of donor material and expensive GMP-compliant expansion of donor cells.

There is consensus in the tissue engineering community that an ideal material should provide for regeneration and not replacement of damaged tissues by attracting endogenous cells to the defect site and instructing them to differentiate via specific cues while at the same time maintaining safety, cost-effectiveness, minimal-invasiveness and a one-step facilitated application during surgery.

It is an aim of certain embodiments of the present invention to at least partially igate the problems associated with the prior art.

It is an aim of certain embodiments of the present invention to provide a material which is capable of delivering multiple therapeutic agents within different regions of the material.

It is an aim of certain embodiments of the present invention to provide a method of producing in vivo scaffold matrices which is biocompatible and low-cost.

### SUMMARY OF CERTAIN EMBODIMENTS OF THE INVENTION

In a broad aspect of the invention, there is provided matrix materials which may encompass biologically active molecules which spatial arrangement may be controlled. Particularly, certain aspects of the present invention are based on a combination of a development of controlled loading of biologically active molecules and a synthesis method for transfection-grade divalent cation/phosphate/nucleic acid (or other biological molecule) nanoparticles within defined areas of the biomaterial to provide a novel platform technology for rapid and cost-effective generation of matrices for non-viral delivery of biologically active molecules in vivo.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991).

In certain embodiments, biocompatible calcium-phosphate nanoparticles (or other divalent cation derived phosphate nanoparticles) not only provide delivery of biologically active molecules such as therapeutically effective genes but are also expected to synergistically direct tissue formation due to their chemical nature, for example, by influencing biomineralisation in the target area, thus improving the efficacy of the overall system. The system may therefore address the challenges associated with the application of materials such as gene-activated matrices and provide a robust low-cost system for technological advance over the current limitations of non-viral gene therapeutics.

In a first aspect of the present invention, there is provided a biocompatible material for delivering a biological molecule to target location, the material comprising:
a) a hydrogel matrix material; and
b) a divalent cation-phosphate nanoparticle and a biological molecule, and further wherein the nanoparticle is encompassed within the hydrogel matrix material.

Aptly, the nanoparticle is associated with a biological molecule.

As used herein, the term “biocompatible material” relates to a material which is suitable for in vivo use. For example, the material is aptly non-toxic to a subject e.g. a mammalian subject when implanted into or otherwise supplied to the subject. The mammalian subject may be a human subject. In certain embodiments, the biocompatible material has an ability to perform its intended function, with the desired degree of incorporation in a host, e.g. a subject, without eliciting any undesirable local or systemic effects in that subject. In certain embodiments, the biocompatible material has the ability to perform as a substrate that will support an appropriate cellular activity, including the facilitation of molecular and mechanical signalling systems, in order to optimise tissue regeneration, without eliciting any undesirable effects in those cells, or inducing any undesirable local or systemic responses in the eventual host.

As used herein, the term “hydrogel matrix material” relates to a material typically composed of a polymeric material, the hydrophilic structure of which renders it capable of holding large amounts of water in its three-dimensional networks. In certain embodiments, the hydrogel matrix material comprises a water-swollen, and cross-linked polymeric network produced by a reaction of one or more monomers. In certain embodiments, e.g. in tissue engineering applications, the hydrogel matrix material is configured to provide an extracellular matrix (ECM) analogue for cell growth, offering a milieu in which to direct cell migration, proliferation and remodel the cellular environment. Aptly, the hydrogel matrix material is a three dimensional material.

Aptly, the hydrogel matrix material is suitable for use as a matrix material in an electrophoretic process e.g. a native gel electrophoretic technique. Suitable materials for a hydrogel matrix material include for example a material selected from hyaluronic acid, polyethylene glycol, agarose, collagen, alginate, chitosan, poly(lactic) acid, poly(lactic-co-glycolic) acid, fibrin, platelet-rich plasma gel and combinations thereof. In certain embodiments, the hydrogel matrix material is a genetic technology grade (GTG) certified material and suitable for use in vivo.

Aptly, the hydrogel matrix material comprises agarose e.g. an agarose gel material. Aptly, agarose is a linear polymer with a MW of about 120,000 isolated from agar or agar-bearing marine algae. Aptly, agarose comprises alternating D-galactose and 3,6-anhydro-L-galactopyranose units. Agarose is widely available. Aptly, in certain embodiments, the agarose is a genetic technology grade (GTG) certified agarose. Such agarose may be available from Lonza for example under the trade names Seakem GTG and SeaPlaque GTG. In certain embodiments, the hydrogel matrix material comprises agarose in a concentration of between about 1% and about 4% w/v. In certain embodiments, the hydrogel matrix material has a gelling temperature of between about 26 to about 28° C. In certain embodiments, the hydrogel matrix material comprises a low-melting point agarose (e.g. an agarose which has a remelting point of 65° C. or lower at a concentration of about 1.5% w/v.

As used herein the term “nanoparticle” and “divalent cation-phosphate nanoparticle” are interchangeable and taken to refer to a nano-sized particles or granules. Aptly, the particles are porous. In certain embodiments, the nanoparticle has a diameter of between about 50 to about 1000 nm. Thus, the nanoparticle has a diameter of e.g. 50, 100, 150, 200, 250, 300, 350, 400, 450, 500, 550, 600, 650, 700, 750, 800, 850, 900, 950 or 1000 nm. The nanoparticles may be spherical in shape. In alternative embodiments, the nanoparticles may be non-spherical in shape e.g. an irregular shape.

Aptly, the nanoparticle is composed of and/or comprises a divalent cation and a phosphate.

In certain embodiments, the divalent cation is selected from Ba²⁺, Co²⁺, Ca²⁺, Mg²⁺ and Sr²⁺. In certain embodiments, the nanoparticle further comprises a branched or linear amine-containing cationic poly-cation. In certain embodiments, the branched or linear amine-containing cationic poly-cation is poly-ethylene imine (PEI). Aptly, the branched or linear amine-containing cationic poly-cation, e.g. PEI has a molecular weight of between about 5 kDa and about 25 kDa.

In certain embodiments, the divalent cation and/or the phosphate complex with the divalent cation has a pharmacological action which acts in addition to the biological molecule. For example, in certain embodiments, CaP, and/or SrP may enhance bone regeneration. Mg²⁺ may be used as an inhibitor of bone mineralisation. In certain embodiments, the nanoparticle may comprise hydroxyapatite.

Optionally, the nanoparticle comprises a [divalent cation]: [phosphate] ratio of less than or equal to 925. Optionally, the nanoparticle comprises a [divalent cation]: [phosphate] ratio of less than or equal to 750.

Optionally, the nanoparticle comprises a [divalent cation]: [phosphate] ratio of less than or equal to 500.

In certain embodiments, the nanoparticle is associated with a biological molecule.

As used herein, the term “associated with” refers to a relationship between the nanoparticle and a biological molecule. The nanoparticles and the biological molecule may be directly or indirectly associated. In certain embodiments, the nanoparticle may form a complex with the biological molecule. Aptly, the nanoparticle partially or wholly encapsulates the biological molecule.

In certain embodiments, the nanoparticle is complexed with the biological molecule. Optionally the biological molecule is a biologically active molecule.

In certain embodiments, the biocompatible material comprises a complex comprising the divalent cation-phosphate and the biological molecule.

In certain embodiments, the biocompatible material comprises a complex comprising the divalent cation-phosphate associated with the biological molecule.

As used herein, the term “biological molecule” refers to a molecule which has a biological activity e.g. activity in vivo or is a precursor to a biologically active molecule. Aptly, the term may be used to refer to a molecule which can be made using biological techniques. In some embodiments, the molecule may be a synthetic molecule which has an effect in vivo e.g. a small molecule compound or the like. Non-limiting examples of a biological molecule include e.g. steroids, peptides and nucleic acids which may be synthesized chemically.

In certain embodiments, the biological molecule is a biologically active molecule. The term biological activity, as used herein, refers to one or more intercellular, intracellular or extracellular process (e.g., cell-cell binding, ligand-receptor binding and cell signalling, etc.) which can impact physiological or pathophysiological processes.

The term “biological molecule” may also be used herein to refer to derivatives of naturally derived molecules, e.g. molecules which have been chemically modified e.g. to add PEG groups or the like.

Non-limiting examples of suitable biological molecules are provided herein.

Aptly, the biological molecule is a charged molecule. Optionally, the biological molecule is a therapeutic agent.

In certain embodiments, the biological molecule is selected from a nucleic acid molecule, a polypeptide and a cell. The nucleic acid molecule may be single-stranded or double-stranded.

As used herein, the term “nucleic acid molecule” refers to deoxyribonucleotide molecules, ribonucleotide molecules, or modified nucleotides, and polymers thereof. The nucleic acid molecule may be in a single- or double-stranded form. The term encompasses a nucleic acid molecule which contains known nucleotide analogs or modified backbone residues or linkages, which are synthetic, naturally occurring, and non-naturally occurring, which have similar binding properties as the reference nucleic acid molecule, and which are metabolized in a similar manner. Examples of such analogs include, without limitation, phosphorothioates, phosphoramidates, methyl phosphonates, chiral-methyl phosphonates, 2-O-methyl ribonucleotides and peptide-nucleic acids (PNAs).

Aptly, a nucleic acid molecule comprises a plurality of nucleotides. As used herein, the term “nucleotide” refers to a ribonucleotide or a deoxyribonucleotide, or a modified form thereof. Nucleotides include species that include purines (e.g., adenine, hypoxanthine, guanine, and the like) as well as pyrimidines (e.g., cytosine, uracil, thymine, and the like). When a base is indicated as “A”, “C”, “G”, “U”, or “T”, it is intended to encompass both ribonucleotides and deoxyribonucleotides, and modified forms thereof.

The nucleic acid molecule may be synthetic or naturally-occurring. The term “naturally occurring” may refer to something found in an organism without any intervention by a person; it could refer to a naturally-occurring wildtype or mutant molecule. A synthetic nucleic acid molecule may be an analogue of a naturally-occurring nucleic acid molecule or may be different.

In certain embodiments, the nucleic acid molecule is selected from a miRNA, an RNA aptamer and a DNA aptamer.

In one embodiment the nucleic acid molecule may be a miRNA. The term “miRNA” is used according to its ordinary and plain meaning and refers to a microRNA molecule found in eukaryotes that is involved in RNA-based gene regulation. See, e.g., Carrington et al., 2003, which is hereby incorporated by reference. The term will be used to refer to the single-stranded RNA molecule processed from a precursor.

In certain embodiments, the nucleic acid molecule is an aptamer. Aptly, the aptamer is an RNA aptamer or a DNA aptamer.

The term “aptamer”, as used herein, refers to a non-naturally occurring nucleic acid that has a desirable action on a target molecule. Desirable actions include, but are not limited to, binding of the target, inhibiting the activity of the target, enhancing the activity of the target, altering the binding properties of the target (such as, for example, increasing or decreasing affinity of the target for a ligand, receptor, cofactor, etc.), inhibiting processing of the target (such as inhibiting protease cleavage of a protein target), enhancing processing of the target (such as increasing the rate or extent of protease cleavage of a protein target), and inhibiting or facilitating the reaction between the target and another molecule. An aptamer may also be referred to as a “nucleic acid ligand.”

In some embodiments, an aptamer specifically binds a target molecule, wherein the target molecule is a three dimensional chemical structure other than a polynucleotide that binds to the aptamer through a mechanism which is independent of Watson/Crick base pairing or triple helix formation, and wherein the aptamer is not a nucleic acid having the known physiological function of being bound by the target molecule. In some embodiments, aptamers to a given target include nucleic acids that are identified from a candidate mixture of nucleic acids, by a method comprising: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers to the target molecule are identified.

An aptamer can include any suitable number of nucleotides. Aptamers may comprise DNA, RNA, both DNA and RNA, and modified versions of either or both, and may be single stranded, double stranded, or contain double stranded or triple stranded regions, or any other three-dimensional structures. In some embodiments, aptamers may be obtained by a technique called the systematic evolution of ligands by exponential enrichment (SELEX) process (Tuerk et al., Science 249:505-10 (1990), U.S. Pat. Nos. 5,270,163, and 5,637,459, each of which is incorporated herein by reference in their entirety).

In certain embodiments, the biological molecule is a double-stranded nucleic acid molecule. Optionally, the double-stranded nucleic acid molecule is selected from siRNA, pDNA, a gene, e.g. a synthetic gene (linear, 5′ and 3′ end-hairpin ligated expression cassette), mRNA e.g. synthetic messenger RNA (mRNA).

The term “siRNA” (short interfering RNA) is a term used in the art and refers to a short double stranded RNA complex, typically 19-28 base pairs in length and which operates in the RNAi pathway where it interferes with the expression of specific genes with complementary nucleotide sequences by degrading mRNA after transcription. Aptly, siRNA is a is double-stranded nucleic acid molecule comprising two nucleotide strands, each strand having about 19 to about 28 nucleotides (i.e. about 19, 20, 21, 22, 23, 24, 25, 26, 27, or 28 nucleotides). The complex often includes a 3′-overhang. SiRNA can be made using techniques known to one skilled in the art and a wide variety of siRNA is commercially available.

In certain embodiments, the biological molecule is selected from:
a) a polypeptide; and
b) a nucleic acid molecule encoding a polypeptide.

Aptly, the nucleic acid molecule is a plasmid or vector encoding a plurality of polypeptides.

The term “vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and aptly, is a DNA plasmid.

In certain embodiments, the biological molecule is a polypeptide.

The terms “polypeptide,” “peptide,” and “protein” are used interchangeably herein to refer to polymers of amino acids of any length. The polymer may be linear or branched, it may comprise modified amino acids, and/or it may be interrupted by non-amino acids. The terms also encompass an amino acid polymer that has been modified naturally or by intervention; for example, by way of disulphide bond formation, glycosylation, lipidation, acetylation, phosphorylation, or any other manipulation or modification, such as conjugation with a labelling component. Also included within the definition are, for example, polypeptides containing one or more analogs of an amino acid (including, for example, unnatural amino acids, etc.), as well as other modifications known in the art. Polypeptides can be single chains or associated chains.

Optionally, the polypeptide or plurality of polypeptides is selected from a growth factor, a cytokine, an antibody, an antibody fragment and an extracellular matrix protein. The protein may be a fusion protein for example.

Examples of extracellular proteins include growth factors, cytokines therapeutic proteins, hormones and peptide fragments of hormones, inhibitors of cytokines, peptide growth and differentiation factors, interleukins, chemokines, interferons, colony stimulating factors and angiogenic factors.

In certain embodiments, the polypeptide is a growth factor selected from basic fibroblast growth factor (bFGF, or FGF-2), acid fibroblast growth factor (aFGF), epidermal growth factor (EGF), heparin binding growth factor (HBGF), fibroblast growth factor (FGF), vascular endothelium growth factor (VEGF), transforming growth factor, (e.g. TGF-α, TGF-β, and bone morphogenic proteins such as BMP-2, -3, -4, -6, -7), Wnts, hedgehogs (including sonic, indian and desert hedgehogs), noggin, activins, inhibins, insulin-like growth factor (such as IGF-I and IGF-II), growth and differentiation factors 5, 6, or 7 (GDF 5, 6, 7), leukemia inhibitory factor (LIF/HILDA/DIA), Wnt proteins, platelet-derived growth factors (PDGF), bone sialoprotein (BSP), osteopontin (OPN), CD-RAP/MIA, SDF-1(alpha), HGF and parathyroid hormone related polypeptide (PTHrP).

In certain embodiments, the polypeptide is selected from TGF-β3, BMP2, BMP6, BMP7, CD-RAP/MIA and combinations thereof.

In certain embodiments, the biological molecule is an extracellular matrix protein, wherein optionally the extracellular matrix protein is selected from collagen, chondronectin, fibronectin, laminin, vitronectin and a proteoglycan.

In certain embodiments, the biological molecule is a cell surface protein. Examples of cell surface proteins include the family of cell adhesion molecules (e.g., the integrins, selectins, Ig family members such as N-CAM and L1, and cadherins); cytokine signaling receptors such as the type I and type II TGF-receptors and the FGF receptor; and non-signaling coreceptors such as betaglycan and syndecan. Examples of intracellular RNAs and proteins include the family of signal transducing kinases, cytoskeletal proteins such as talin and vinculin, cytokine binding proteins such as the family of latent TGF-binding proteins, and nuclear trans acting proteins such as transcription factors and enhancing factors.

In certain embodiments, the biological molecule is a nucleic acid molecule e.g. a gene which encodes a protein as described herein. Aptly, the nucleic acid molecule encodes an extracellular protein e.g. a growth factor, a cytokine, a therapeutic protein, a hormone. Aptly, the nucleic acid molecule encodes a peptide fragment of a hormone, an inhibitor of cytokines, peptide growth and differentiation factor, an interleukin, a chemokine, an interferon, a colony stimulating factor or an angiogenic factor.

In certain embodiments, the biological molecule may be a conjugate e.g. an “immunoconjugate”. As used herein, the term “immunoconjugate” is an antibody conjugated to one or more heterologous molecule(s), including but not limited to a cytotoxic agent.

In certain embodiments, the biological molecule is a cell, and wherein the cell is selected from a neural cell (e.g. a neuron, a oligodendrocytes, a glial cell, an astrocyte), a lung cell, a cell of the eye (e.g. a retinal cell, a retinal pigment epithelial cell, a corneal cell), an epithelial cell, a muscle cell, a bone cell (e.g, a bone marrow stem cell, an osteoblast, an osteoclast or an osteocyte), an endothelial cell, a hepatic cell and a stem cell.

In certain embodiments, the biocompatible material comprises a plurality of divalent cation-phosphate nanoparticles, wherein the plurality of divalent cation-phosphate nanoparticles are dispersed within the hydrogel matrix material.

Aptly, the plurality of divalent cation-phosphate nanoparticles comprises a first set of divalent cation-phosphate nanoparticles having a first predetermined spatial distribution with respect to the hydrogel matrix material and a further set of divalent cation-phosphate nanoparticles having a further pre-determined spatial distribution with respect to the hydrogel matrix material.

Certain embodiments of the present invention provide a material in which the spatial distribution of a plurality of biological molecules e.g. those associated with a nanoparticle as described herein may be controlled. Aptly, the material is three dimensional. As used herein, the term “spatial distribution” can refer to distribution of the nanoparticles and/or biological molecule in an x-direction, a y-direction and/or a z-direction within the material. The biological molecules and/or nanoparticles may be evenly distributed within the material. Alternatively, the material may comprise a region which comprises nanoparticle/biological molecules in a higher concentration than a further region of the region.

In certain embodiments, the first predetermined spatial distribution differs from the further predetermined spatial distribution. Aptly, the first predetermined spatial distribution and/or the further predetermined spatial distribution each create a concentration gradient of the biological molecule and/or nanoparticle distribution.

In certain embodiments, the plurality of divalent cation-phosphate nanoparticles comprises a first set of divalent cation-phosphate nanoparticles and a further set of divalent cation-phosphate nanoparticles, wherein the nanoparticles of the first set comprise at least one predetermined characteristic and the nanoparticles of the further set comprise at least one further predetermined characteristic.

Optionally, the first set of divalent cation-phosphate nanoparticles differs in at least one characteristic from the further set of divalent cation-phosphate nanoparticles. Optionally, the at least one first characteristic and the at least one further characteristic are independently selected from:
a) particle size;
b) type of divalent cation;
c) type of biological molecule;
d) rate of biological molecule release;
e) concentration of biological molecule; and
f) a combination of (a) to (e).

In certain embodiments, the plurality of nanoparticles comprise an average diameter of between about 50 to about 1000 nm. Thus, the nanoparticle has a diameter of e.g. 50, 100, 150, 200, 250, 300, 350, 400, 450, 500, 550, 600, 650, 700, 750, 800, 850, 900, 950 or 1000 nm.

In certain embodiments, a first subset of nanoparticles may be associated with a first biological molecule and a further subset of nanoparticles may be associated with a further biological molecule. Thus, the material may comprise a plurality of first biological molecules e.g. a nucleic acid molecule, a protein and/or a cell as described herein and further comprise a plurality of further biological molecules e.g. a nucleic acid molecule, a protein and/or a cell as described herein. Aptly, the first subset of nanoparticles may be provided in a first zone of the material and the further subset of nanoparticles may be provided in a further zone of the material. Aptly, the first zone and further zone may be the same zone or may be different. Optionally, the material may comprise two, three, four, five or more different types of biological molecules, wherein aptly each biological molecule is associated with a nanoparticle.

Thus, certain embodiments of the present invention provide a material which is suitable for delivering a plurality of biological molecules to a location in vivo wherein the plurality of biological molecules may replicate the complex in vivo cellular environment.

In certain embodiments, the material may enable localized, sustained transgene expression to be achieved, which promotes the expression of growth factors directly within the local environment and eventually tissue formation.

In certain embodiments, the material may provide simultaneous or sequential delivery of multiple biological molecules.

In certain embodiments, the biocompatible material further comprises a bioactive agent. The bioactive agent may be a molecule which is the same as the biological molecule as described herein. Alternatively, the bioactive agent may be a different molecule to the biological molecule. Aptly, the bioactive agent is a polypeptide, for example, an extracellular matrix protein e.g. fibronectin, laminin and/or heparin. In certain embodiments, fibronectin as an additive can increase gene transfer efficacy. In certain embodiments, fibronectin may improve uptake of fibronectin containing nanoparticles.

In a second aspect of the present invention, there is provided a three-dimensional scaffold comprising the biocompatible material according to the first aspect of the present invention.

In certain embodiments, the scaffold comprises a plurality of divalent cation-phosphate nanoparticles, wherein the plurality of divalent cation-phosphate nanoparticles comprises a first set of divalent cation-phosphate nanoparticles and a further set of divalent cation-phosphate nanoparticles, and
further wherein the nanoparticles of the first set comprise at least one predetermined characteristic and the nanoparticles of the further set comprise at least one further predetermined characteristic,
and further wherein the scaffold comprises a first zone and a further zone, said first zone comprising a majority of the first set of divalent cation-phosphate nanoparticles and the second zone comprising a majority of the second set of divalent cation-phosphate nanoparticles.

Optionally the first set and the second set differ in at least one predetermined characteristic. In certain embodiments, the first zone is a first end of the scaffold and the further zone is a further end of the scaffold.

Aptly, the further zone is a second zone and the scaffold further comprises a third zone, and further wherein the third zone is provided between the first zone and the second zone.

In certain embodiments, the scaffold is loaded with one or more cells. The cells may be loaded to an external surface of the scaffold. Aptly, the one or more cells may be of the same or differing types. For example, the one or more cells may be selected from a neural cell (e.g. a neuron, a oligodendrocytes, a glial cell, an astrocyte), a lung cell, a cell of the eye (e.g. a retinal cell, a retinal pigment epithelial cell, a corneal cell), an epithelial cell, a muscle cell, a bone cell (e.g. a bone marrow stem cell, an osteoblast, an osteoclast or an osteocyte), an endothelial cell, a hepatic cell and a stem cell.

In certain embodiments the first set of divalent cation-phosphate nanoparticles are associated with a biological molecule which is chondrogenic. The term “chrondrogenic” refers to causing or having a role in the development of cartilage. In certain embodiments, the biological molecule is a polypeptide having chrondrogenic properties.

In certain embodiments, the biological molecule is a polypeptide selected from BMP-6, BMP-7, TGF-β3, CD-RAP/MIA and combinations thereof or a nucleic acid encoding a polypeptide selected from BMP-6, BMP-7, TGF-β3, CD-RAP/MIA and combinations thereof.

Optionally, the first set of divalent cation-phosphate nanoparticles are associated with a biological molecule which is osteogenic i.e. is associated with or has a role in the development of a tissue which is involved in bone growth or repair.

In certain embodiments, the biological molecule is a polypeptide selected from BMP-2 and BMP-7 and combinations thereof, and/or heterodimeric BMP e.g. BMP2/6 or BMP4/7 or a nucleic acid molecule encoding a polypeptide selected from BMP-2 and BMP-7 and combinations thereof, and/or heterodimeric BMP e.g. BMP2/6 or BMP4/7.

In a further aspect of the present invention, there is provided a biocompatible material as described herein for use as an in vivo delivery vehicle.

In a further aspect of the present invention, there is provided a three-dimensional scaffold as described herein for use as an in vivo delivery vehicle.

Aptly, the in viva delivery vehicle is for use as a vaccine composition, wherein the biological molecule is an immunogenic molecule or an antigen-encoding nucleic acid molecule.

Aptly, the in vivo delivery vehicle is for use to treat a wound in a subject e.g. a wound site. A wound site may be defined as any location in the subject that arises from traumatic tissue injury, or alternatively, from tissue damage either induced by, or resulting from, surgical procedures. Aptly, the delivery vehicle may be used for bone repair, cartilage repair, tendon repair, ligament, repair, blood vessel repair, skeletal muscle repair, and/or skin repair.

In certain embodiments, the delivery vehicle comprises a biological molecule such as for example an angiogenic factor. Exemplary angiogenic factors include for example vascular endothelial growth factor (VEGF), a platelet-derived growth factor (PDGF) e.g. PDGF13 or a fibroblast growth factor (FGF). Aptly, the angiogenic factor is a human angiogenic factor.

In other embodiments, the biological molecule may be a nucleic acid molecule encoding an angiogenic factor.

Optionally, the in vivo delivery vehicle is for use to regenerate bone and/or cartilage in a subject.

In certain embodiments, the material may deliver multiple growth factors, which may synergistically promote, for example, enhanced angiogenesis and bone regeneration.

In certain embodiments, the scaffold provides a biological molecule in an effective amount.

As used herein, an “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a biological molecule as described herein, or an amount of a combination of biological molecules and/or bioactive agents as described herein, e.g., that is effective to treat or prevent a disease or disorder, or to treat the symptoms of the disease or disorder, in a subject. Thus, an “effective amount” generally means an amount that provides the desired effect.

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

In a further aspect of the present invention, there is provided a vaccine composition comprising the biocompatible material as described herein and/or the three-dimensional scaffold as described herein, wherein the biological molecule is an immunogenic molecule or an antigen encoding nucleic acid molecule.

Aptly the vaccine composition is for oral administration. In certain embodiments, the vaccine composition is for subcutaneous and/or intramuscular administration. Optionally, the immunogenic molecule is provided in a concentration sufficient to induce an immune response in a subject. Aptly, the vaccine composition further comprises an adjuvant molecule.

In a further aspect of the present invention, there is provided a method of treating a wound in a subject, the method comprising:
a) administrating a biocompatible material or a scaffold as described herein to a subject.

In certain embodiments, the method comprises administrating the biocompatible material or scaffold subcutaneously and/or intramuscularly.

In a further aspect of the present invention, there is provided a method of treating a bone defect in a subject, the method comprising:
a) administrating a biocompatible material or a scaffold as described herein to a subject.

In certain embodiments, the method comprises administrating the biocompatible material or scaffold subcutaneously and/or intramuscularly. Aptly, the bone defect is a bone fracture.

In a further aspect of the present invention, there is provided a method of preparing a biocompatible material, the biocompatible material comprising:
a) a hydrogel matrix material;
b) a divalent cation-phosphate nanoparticle,
c) a biological molecule, wherein the nanoparticle and the biological molecule are encompassed within the hydrogel matrix material,
and wherein the method comprises:
i) providing a hydrogel matrix material disposed between a cathode and an anode;
ii) supplying phosphate ions to the hydrogel matrix material;
iii) supplying a solution comprising a biological molecule to the hydrogel matrix material;
iv) supplying a solution comprising a divalent cation to the hydrogel matrix material; and
v) applying an electrical field to the hydrogel matrix material between the cathode and the anode such that a divalent cation-phosphate nanoparticle associated with a biological molecule is formed within the hydrogel matrix material.

In certain embodiments, the phosphate ions are comprised in a buffer solution and step (ii) comprises supplying the buffer solution to the hydrogel matrix material.

In certain embodiments, the method further comprises step (vi) of supplying a buffer solution to the hydrogel matrix material.

In certain embodiments, steps (i) to (iv) and (vi) may be performed in any order.

In certain embodiments, the method comprises suppling a plurality of solutions comprising a biological molecule, wherein at least a first solution of the plurality of solutions comprises a biological molecule which is a different biological molecule to a biological molecule comprised in a further solution of the plurality of solutions.

In certain embodiments, the method comprises supplying the first solution comprising a biological molecule to a first target location in the hydrogel matrix material and wherein the method further comprises supplying the further solution comprising a biological molecule to a further target location within the hydrogel matrix material.

In certain embodiments, the method comprises suppling a plurality of solutions comprising a divalent cation, wherein at least a first solution of the plurality of solutions comprises a divalent cation which is a different divalent cation to a divalent cation comprised in a further solution of the plurality of solutions.

In certain embodiments, the method comprises supplying the first solution comprising a divalent cation to a first target location in the hydrogel matrix material and wherein the method further comprises supplying the further solution comprising a divalent cation to a further target location within the hydrogel matrix material.

In certain embodiments, the method comprises:
supplying the first solution comprising a divalent cation to an anode-facing region of the hydrogel matrix material; and
supplying the further solution comprising a divalent cation to a cathode-facing region of the hydrogel matrix material.

In certain embodiments, the method comprises:
supplying a plurality of solutions comprising a biological molecule, wherein at least a first solution of the plurality of solutions comprises a biological molecule which is a different biological molecule to a biological molecule comprised in a further solution of the plurality of solutions; and
supplying a plurality of solutions comprising a divalent cation, wherein at least a first solution of the plurality of solutions comprises a divalent cation which is a different divalent cation to a divalent cation comprised in a further solution of the plurality of solutions, wherein each of the plurality of solutions comprising a biological molecule and each of the plurality of solutions comprising a divalent cation are supplied to a common region of the hydrogel matrix material, and further wherein the method further comprises alternating the polarity of the electric field such that each of the divalent cations and each of the biological molecules move to a common target location in the hydrogel matrix material.

In certain embodiments, the buffer solution in the gel and electrophoresis system is a cell and DNA-compatible buffer solution. Aptly, the buffer solution is a non-TRIS containing buffer solution. Optionally, the buffer solution is HEPES.

In certain embodiments, the method is carried out under non-denaturing conditions.

In certain embodiments, the method further comprises removing the hydrogel matrix material from an electrophoretic apparatus so as to provide the biocompatible material.

In certain embodiments, the method further comprises soaking or coating the hydrogel matrix material with an extracellular matrix molecule for example fibronectin and laminin and other RGD-sequence containing peptides to enhance cellular attachment.

In certain embodiments, the method further comprises supplying e.g. a plurality of cells to the hydrogel matrix material.

In certain embodiments, the method further comprises lyophilising the hydrogel matrix material to form the biocompatible material.

In certain embodiments, the method further comprises drying the hydrogel matrix material under supercritical drying conditions to form the biocompatible material, wherein the biocompatible material is an aerogel.

In certain embodiments, the method further comprises melting the hydrogel matrix material to form an injectable biocompatible material, wherein the biocompatible can be delivered in a gelled state or the material forms a hydrogel after implantation. Aptly, the agarose is a low melt agarose. Aptly, the agarose has a melting point of approximately 66° C. or below.

In certain embodiments, the method comprises supplying the biological molecule e.g. a nucleic acid molecule at a concentration of up to about 125 μg/cm³. In certain embodiments, the biological molecule is supplied in non-continuously e.g. in pulses.

### DESCRIPTION OF THE FIGURES

Certain embodiments of the present invention are described in more detail below, by way of example only, and with reference to the accompanying drawings in which:

FIG. 1: SEM back-scatter images of lyophilised agarose GAMs and calcium phosphate nanoparticles at a Ca:P ratio of 166.67x. Scale bars represent 20 μm (left) and 10 μm (right);

FIG. 2: SEM back-scatter images of aerogel agarose GAMs and calcium phosphate nanoparticles at a Ca:P ratio of 166.67x. Scale bars represent 50 μm (left) and 10 μm (right).

FIG. 3: Overlay image of calcium phosphate (light blue) and ethidium-bromide stained plasmid DNA (magenta, loaded for 5 min at 60V) in gels after complexation using different ratios of Ca:P (Ca2+ loaded using 60V and reversed polarity). The extent of co-localisation/co-precipitation is observable in dark blue colour in the overlay image;

FIG. 4: Migration of 10 μg of bovine plasma fibronectin in native agarose gel electrophoresis at 60 Volts for different electrophoresis durations (Coomassie staining);

FIG. 5: Fluorescent microscopy images of GFP-positive cells transfected by agarose-GAMs without fibronectin (METHOD1) at a calcium to phosphate ratio of 120.37-fold, 1 week post seeding. Scale bars represent 60.8 μm (left) and 105 μm (right);

FIG. 6: Metridia luciferase activity of supernatant samples taken from cultures containing lyophilised agarose GAMs using different calcium:phosphate complexation ratios (0, 83.33-fold, 120.37-fold, 157.41-fold, 166.67-fold) taken at 48 hours (A); 1 week (B) and 4 weeks (C) post seeding, comparing samples without (left section of graphs) or with (right section of graphs) the addition of bovine fibronectin. *p<0.5, **p<0.01;

FIG. 7: Alkaline phosphatase activity in 02012 cells after incubation with recombinant BMP-containing agarose matrices and control matrices using a Ca:P ratio of 166.67-fold. **p≤0.01 for statistical significance; and

FIG. 8:

Representative bioluminescence image of GAM-induced luciferase expression in vivo with quadrants used for quantification of individual implants (4 per animal) outlined (A). (B) Quantification of CBR luciferase activity in different calcium-phosphate containing groups (FN: fibronectin, CaP: calcium phosphate). (C) Comparison of gene transfer efficacy (CBR luciferase activity) of calcium-phosphate (CaP) containing GAMs with magnesium-phosphate (MgP) containing GAMs and GAMs without nanoparticle complexation. *p≤0.05 (Tukey's multiple comparison test).

FIG. 9: Confocal laser scanning microscopy image of multiple pDNA gradient within hydrogels. pDNA1, 2, 3 were labelled with cyanine dimer dyes and imaged after sequential loading (pDNA1, 2, 3 in sequence; 5 min loading each, total electrophoresis time indicated below individual images). (A) YOYO1 stained pDNA1, (B) POPO3 stained pDNA2, (C) TOTO3 stained pDNA3; Composite image of all 3 channels (D). Scale bar represents 600 μm.

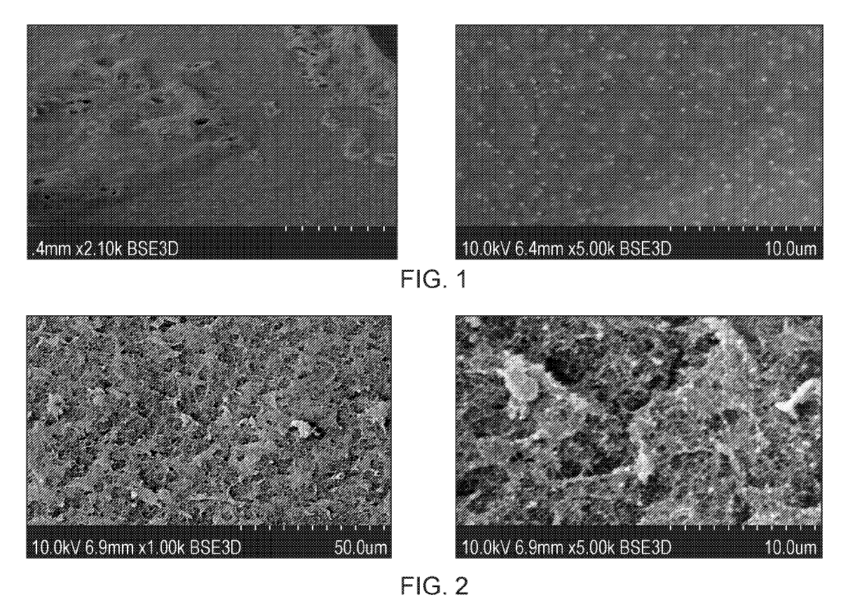

### EXAMPLES

### Example 1

Production of Agarose Gene-Activated Matrices and Gene Delivery In Vitro

In order to demonstrate that electrophoretically-loaded agarose gene-activated matrices (GAMs) can indeed deliver nucleic acids and to investigate the potential beneficial effect of calcium phosphate nanoparticles on gene delivery by the matrix, agarose was loaded with plasmid DNAs (pDNA) encoding luciferase (Metridia luciferase) and green fluorescent protein (GFP) reporter genes using electrophoresis and subsequently agarose-embedded pDNA was complexed with different ratios of calcium (Ca²⁺):phosphate (HPO₄²⁻) ions in order to generate calcium phosphate/DNA co-precipitates during electrophoresis.

1.1 Material and Methods

1.1.1 Matrix Preparation:

METHOD 1: 1% (weight/volume-percent, w/v) agarose matrices (NuSieve 3:1 Agarose, Lonza) were prepared using HEPES buffer (25 mM, 70 mM NaCl, pH 7.05) containing 0.75 mM Na₂HPO₄ and left to solidify at room temperature. Subsequently, solidified agarose gels were submerged in HEPES buffer (same as above) and 2.5 μg each of Metridia luciferase encoding pDNA (pMetLuc Reporter, Clontech) and green fluorescent protein encoding pDNA (pGFPmax, Amaxa) were loaded to gel slots and electrophoresis was performed for 5 minutes at 60 Volts (constant voltage, variable amperage setting). After DNA loading, different amounts of 500 mM CaCl₂) solution were loaded though the same slots in order to generate a range of different theoretical complexation ratios of buffer phosphate amount (constant 0.75 mM buffer) and Ca²⁺ amounts, ranging from 2.25 μmol; 3.25 μmol, 4.25 μmol up to 4.5 μmol (resulting in Ca²⁺:HPO₄²⁻ ratios of 83.3-fold, 120.37-fold, 157.41-fold and 166.67-fold). Complexation was performed using reverse polarity for 5 minutes at 60 Volts. After complexation DNA/Calcium phosphate bands were excised using a scalpel and individual agarose scaffolds were frozen at −86° C. and then lyophilised overnight at 0.0010 millibars (Christ Alpha 2-4 LD_{Plus} lyophiliser). Control samples containing only DNA were obtained in the same way but excised directly after the first loading step and lypohilised as described above for complexed samples. All samples were sterilised by incubation in 70% ethanol for 24 h and lyophilised again to remove ethanol. Given that agarose electrophoresis was carried out without the use of a DNA dye, successful band excision was confirmed by post-staining the remaining gel using 0.5 μg/ml Ethidium Bromide containing electrophoresis buffer for staining for 15 min at room temperature and confirming the lack of remaining pDNA at the excision sites.

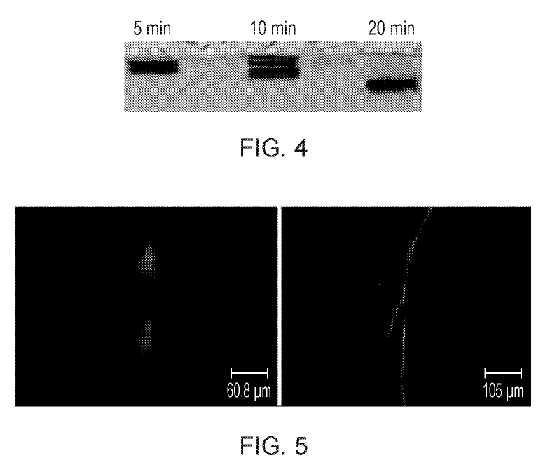

METHOD 2: Additionally, agarose matrices containing DNA and bovine plasma fibronectin (Gibco) were prepared by loading 2.5 μg Metridia encoding plasmid DNA (pMetLuc Reporter) and 2.5 μg green fluorescent protein encoding plasmid DNA (pGFPmax, Amaxa) simultaneously with 10 μg fibronectin under non-denaturing conditions. As fibronectin has been shown to be negatively charged under native conditions in agarose electrophoresis using the same conditions as above (see FIG. 4), it was anticipated that fibronectin would co-migrate with the pDNA under the chosen conditions. DNA/fibronectin loading was carried out for 5 min at 60 Volts under the same conditions as standard samples without fibronectin. Complexation with calcium phosphate was performed in parallel to the protocol used for samples without fibronectin used above. After complexation DNA/Calcium phosphate bands and control samples containing only pDNA were excised using a scalpel and individual agarose scaffolds were frozen at −86° C. and lyophilized overnight. Successful excision of DNA/fibronectin containing gel pieces was confirmed as performed previously (see above).

METHOD 3: For multi-gene distribution imaging purposes, agarose matrices containing multiple different plasmid DNAs were prepared by loading 5 μg each of different plasmid DNAs (pMetLuc Reporter, pGFPmax and pCBR) after staining the pDNAs with cyanine dimer dyes (YOYO1, POPO3 and TOTO3 respectively) before loading onto the gels. pDNAs were loaded sequentially (5 min intervals) at 60 Volts under the same conditions as standard samples.

1.1.2 Matrix Characterisation In Vitro:

Scanning Electron Microscopy (SEM)-Characterisation

For SEM evaluation, agarose matrix samples prepared with calcium:phosphate ratios of 166.67-fold using METHOD1 were either lyophilised to produce lyophilised matrices or supercritical point dried after buffer exchange for acetone using CO₂ to produce aerogels. Samples were sputter coated with gold using an Agar Auto Sputter Coater (approximately 10 nm layer thickness) and then imaged on a Hitachi 53400N scanning electron microscope using dry-stage, back-scattered electron imaging at a beam accelerating voltage of 10 kV, to enable imaging of calcium phosphate precipitates within the matrices (FIG. 1).

DNA and Calcium Phosphate Co-Precipitation

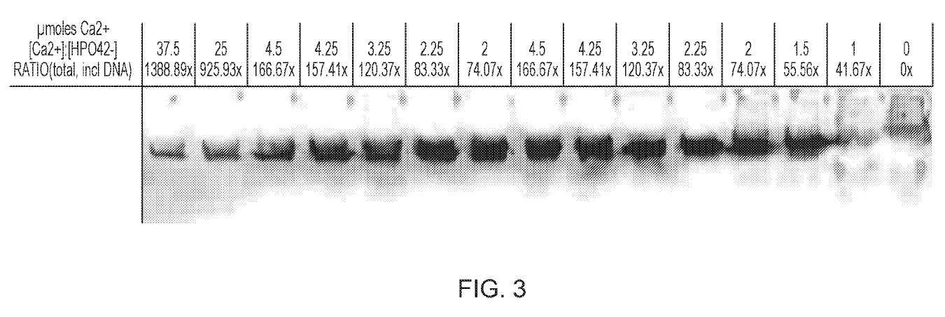

Direct observation of DNA/Calcium phosphate precipitation at different complexation-ratios was carried out in parallel using gels prepared with METHOD1 and a wider range of loaded Ca²⁺ amounts but additionally post-stained with Ethidium Bromide (0.5 μg/ml, 15 min) and imaging of pDNA-localisation was performed after electrophoresis via UV transillumination and calcium phosphate precipitation was imaged using standard VIS imaging (Biorad ChemiDoc MP Imaging System, Image Lab Software). Overlays were produced assigning Ethidium-bromide stained pDNA the red (magenta) and calcium phosphate the blue channel in merged images (FIG. 3).

Confocal Laser Scanning Microscopy of Multiple pDNA Gradients within Hydrogels

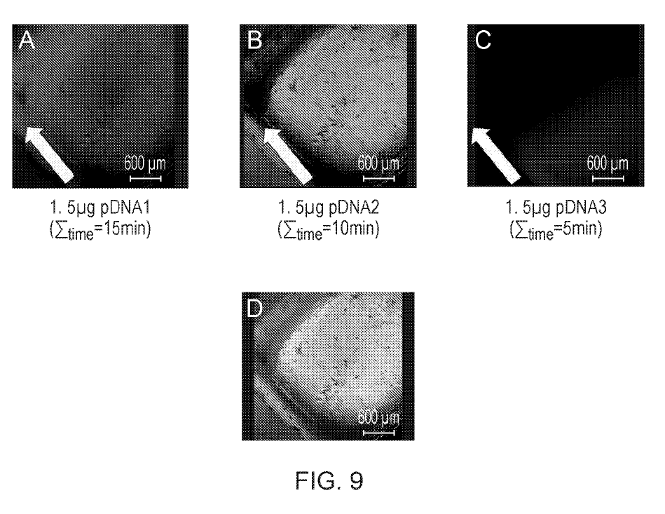

Hydrogel samples were excised after loading and slices were used for confocal microscopy (CLSM) imaging of the obtained gradients of 3 different pDNAs within the matrices (FIG. 9). DNA bands within gels were detected using CLSM with multi-channel detection at specified wavelengths (YOYO1: 509 nm, POPO3: 574 nm, TOTO3: 660 nm).

In Vitro Transfection

The matrices prepared by METHOD1 and METHOD2 for cell culture were preconditioned with 100 μl of DMEM for 2 hours prior to seeding. Then 5×10⁴ C2C12 cells were seeded onto the scaffolds in a 96-well plate in 15 μl DMEM for 2 hours and subsequently supplemented with 200 μl growth medium (DMEM containing 4.5 g/L glucose, 5% fetal bovine serum, 4 mML-glutamine and 1% penicillin/streptomycin) and cultured at 37° C., 5% CO₂, humidified atmosphere in the cell culture incubator for up to 4 weeks. Supernatants containing the secreted luciferase reporter gene were sampled at 48 hours, 1 week and 4 weeks post seeding for gene expression monitoring and where possible microscopic images of GFP fluorescent cells were taken (FIG. 5).

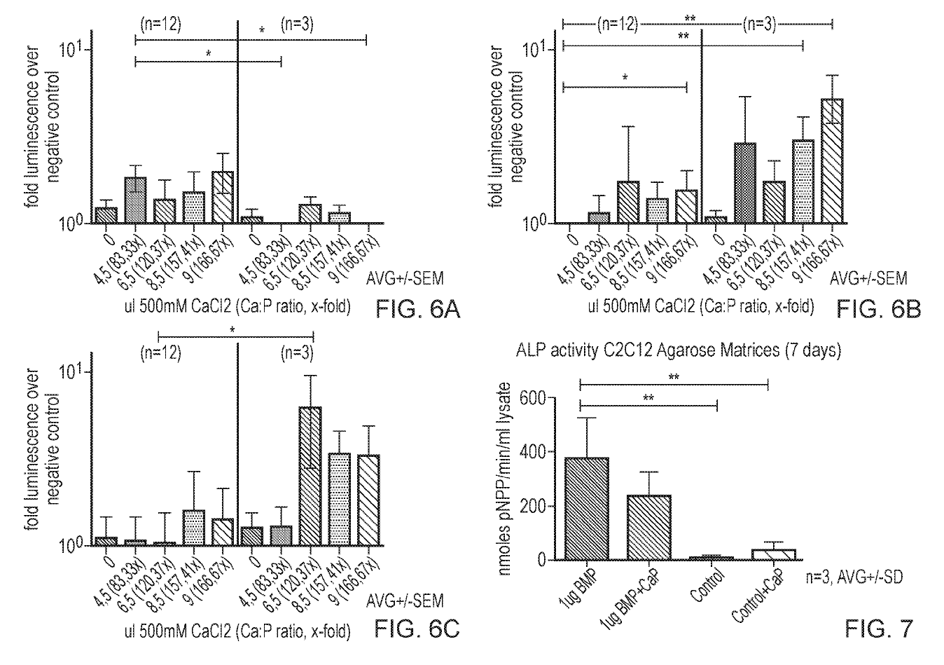

Metridia luciferase activity was determined using coelenterazine provided as a kit using the manufacturer's instructions (Ready-To-Glow™ protocol, Clontech) and quantified in a Varioskan Flash plate luminometer using white 96-well plates. Metridia luciferase activity was calculated in fold-activity compared to agarose GAM control matrices containing only DNA without calcium phosphate precipitation (FIG. 6).

1.2 Results

1.2.1 Matrix Characterisation In Vitro

SEM-Characterisation

SEM-imaging of agarose matrices demonstrated the formation of calcium phosphate nanoparticles in pDNA containing gels and the possibility of producing lyophilised gels and aerogels with different surface topologies through different processing routes (FIG. 1).

DNA and Calcium Phosphate Co-Precipitation

The complexation study demonstrated that the chosen loading/complexation strategy using pDNA loaded to a phosphate containing gel via electrophoresis and then applying CaCl₂ solution for loading Ca²⁺ through the same slots in an electric field of reversed polarity leads to precipitation of calcium phosphate and the co-localisation/co-precipitation of this calcium phosphate with pDNA (FIG. 3). [Ca²⁺ ]: [HPO₄²⁻] ratios ≥83.33-fold lead to complete immobilisation of the pDNA and co-localisation with the bulk of calcium phosphate precipitate. Very high [Ca²⁺]: [HPO₄²⁻] ratios of ≥925 lead to an increase in calcium phosphate precipitation in the gel but a marked reduction in co-localisation/co-precipitation of pDNA with the calcium phosphate particles.

In Vitro Transfection

The result provided herein demonstrate that it is possible to use the material described herein to deliver biological molecules e.g. nucleic acid molecules and that DNA can be delivered from such systems effectively into cells in vitro as observed by fluorescence microscopy for GFP 1 week post seeding and using detailed quantification of gene delivery efficacies via luciferase measurements. In fact, the complexation of pDNA within the gel with calcium phosphate nanoparticles significantly increased the Metridia luciferase activity-associated gene transfer efficacy 1 week post seeding for both GAM matrix systems with nanoparticles produced by METHOD1 and METHOD2 compared to matrices only containing naked pDNA (FIGS. 4 and 5, from 1.6-fold up to 5.3-fold respectively).

Furthermore, there was an additional significant enhancement of gene transfer efficacy observed in matrices containing fibronectin (prepared by METHOD2) when compared to matrices at the same calcium:phosphate complexation ratio (prepared by METHOD1) at 4 weeks post seeding (FIG. 5 and FIG. 6C, up 6.13-fold).

Generally, there was a trend to higher gene delivery efficacies at later timepoints in fibronectin containing matrices, indicating a difference in release/transfection kinetics and beneficial effect of fibronectin on gene delivery in matrices containing nanoparticles. There was however, no beneficial effect observed if fibronectin was added to matrices without calcium phosphate nanoparticles.

This data clearly demonstrates the capability of the method to produce transfection-capable GAMs and to enhance their transfection efficacy by the additional complexation with calcium phosphate nanoparticles during electrophoresis and demonstrates the beneficial effects of adding fibronectin (compatible with the electrophoretic approach using native electrophoresis of negatively charged fibronectin) to the system.

Confocal Laser Scanning Microscopy of Multiple pDNA Gradients within Hydrogels

CLSM showed the establishment of different zones containing different pDNAs within the hydrogel, demonstrating the capability of the developed method to generate matrices with distinct spatial distribution of therapeutic payloads using sequential electrophoretic loading. It was possible to detect each of the 3 different pDNAs within the gels using cyanine dimer labelling and DNA distribution and gradient formation was dependent on the sequence of loading and total loading time for each of the 3 pDNAs (FIG. 9).

### Example 2: Production of Agarose Matrices for Recombinant Protein Delivery In Vitro

The ability of the material described herein to act as a matrix for biologically active recombinant growth factor molecules was investigated. Particularly, it was investigated whether such molecules could also be loaded to agarose matrices, preserving their bioactivity and to use such recombinant growth factor containing matrices for the directed differentiation of target cells in vitro and if the additional formation of calcium phosphate nanoparticles would influence the extend of differentiation of target cells.

2.1 Material and Methods

2.1.1 Matrix Preparation

METHOD: Agarose matrices were prepared according to METHOD1 in Example 1 but instead of pDNA, 1 μg of recombinant human bone morphogenetic protein 2 (rhBMP2, CHO-derived, PeproTech) was loaded during the first round of electrophoresis (60V, 20 min, standard polarity) after protein loading, samples were either subjected to calcium phosphate particle precipitation (60V, 5 min reversed polarity, [Ca²]: [HPO₄²⁻] ratio 166.67-fold) or used without additional nanoparticles. Growth-factor free matrices with or without nanoparticles were used as controls. The matrices were processed as described in Example 1, METHOD1.

2.1.2 In Vitro Differentiation Assay

24 h post preparation and processing; 5×10⁴ C2C12 cells were seeded onto the scaffolds in a 24-well plate in 200 μl DMEM for 2 hours and subsequently supplemented with 1 ml differentiation assay medium (DMEM containing 4.5 g/L glucose, 1% fetal bovine serum, 4 mM L-glutamine and 1% penicillin/streptomycin) and cultured at 37° C., 5% CO₂, humidified atmosphere in the cell culture incubator for 7 days. On day 7 the matrices were removed and the cell lawn was washed once with 1× phosphate buffered saline (PBS) and then washed once with alkaline-phosphatase (ALP) assay buffer. The cells were lysed with 100 μl lysis buffer (ALP-buffer containing 0.25% Triton X-100) on room temperature for 1 h on a plate shaker and then 100 μl of ALP-buffer containing 7.4 mg/ml (20 mM) p-Nitrophenyl phosphate (pNPP) was added and the plate was incubated for 20 min in the dark at 37° C. The samples were then transferred to sterile Eppendorf tubes, centrifuged at 13.000 rpm for 2 min and then 100 μl of cleared lysate/reaction mix were measured at 405 nm on a plate reader (Varioskan Flash). The obtained optical densities (OD₄₀₅) and a standard curve were used to calculate the amount of the released ALP-enzyme reaction product p-Nitrophenol per minute, which gives a direct indication of the extent of osteogenic differentiation induced by rhBMP2 in C2C12 cells.

2.2 Results

2.2.1 In Vitro Differentiation Assay

ALP-activity assays demonstrated that it is possible to use the described electrophoretic approach to load bioactive molecules to agarose matrices and that these molecules retain their biological activity even after processing of the gels and thus can be used to deliver growth factors. The recombinant protein rhBMP2 used in this study clearly induced osteogenic differentiation in C2C12 cells after 7 days of exposure to the rhBMP2 containing matrices as observed by significantly elevated ALP-activity. There was no significant increase in ALP activity observable in the growth-factor free controls.

### Example 3: Gene Delivery In Vivo Using Agarose Gene-Activated Matrices

3.1 Material and Methods

3.1.1 GAM Preparation

GAMs for in vivo implantation were prepared using similar protocols as for in vitro GAMs (see above) but contained an increased amount of pDNA (25 μg). The matrices were prepared at a calcium:phosphate ratio of 166.67-fold of loaded Ca2+ to phosphate buffer. Magnesium phosphate containing matrices were also investigated in this study, employing the same complexation ratio and preparation method as described for the calcium-phosphate nanoparticle containing matrices.

Matrices were loaded using 60V for 5 min for pDNA (for the in vivo studies a red-shifted click beetle luciferase, CBR in the plasmid pCBR Control (Promega) was used) loading and 60V for 5 min reversed polarity for complexation. GAMs were either prepared without addition of fibronectin (METHOD1) or with the addition of 10 μg of bovine fibronectin during the pDNA loading step (METHOD2). After complexation DNA/Calcium phosphate or DNA/Magnesium phosphate bands obtained by METHOD1 and METHOD2 were excised using a scalpel and individual agarose scaffolds were frozen at −86° C. and then lyophilised overnight at 0.0010 millibars (Christ Alpha 2-4 LDPlus lyophiliser). All samples were sterilised by incubation in 70% Ethanol for 24 h and lyophilised again to remove ethanol.

Control samples containing only pDNA were obtained in the same way but excised directly after the first loading step and lypohilised as described above for complexed samples.

3.1.2 In Vivo Implantation

24 h post preparation the matrices were subcutaneously implanted in the backs of male outbred MF-1 mice (5 weeks, 25-30 g, Charles River) under inhalation anaesthesia (Isoflurane 3% for induction, 1.5% for maintenance, 1 L/min 02) and pockets were closed using resorbable sutures (VICRYL*rapide, polyglactin 910, Ethicon; Johnson & Johnson). 4 samples were implanted per animal (resulting in 4 imaging quadrants) and samples of the different groups (only pDNA, pDNA+fibronectin, pDNA+calcium phosphate, pDNA+calcium phosphate+fibronectin, pDNA+magnesium phosphate) were applied in a randomised, blocked design. Animals received 0.125 mg/kg buprenorphine (Vetergesic, Alstoe Veterinary) for analgesia intraoperatively as subcutaneous injection. Postoperative antibiosis was administered for 1 week using Baytril® 0.25 mg/ml (Enrofloxacin, Bayer HealthCare Animal Health Division) in the drinking water provided ad libitum.

3.1.3 In Vivo Bioluminescence Imaging

In vivo CBR-luciferase activity was imaged on a Xenogen IVIS imaging station 2 weeks post implantation. Animals each received a 100 μl injection of 5 mg D-luciferin potassium salt (Promega) in physiologic NaCl intraperitoneally prior to imaging and bioluminescence was quantified using the Living Image Software on the imaging station approx. 15 min post injection.

3.2 Results

3.21. In Vivo Bioluminescence Imaging

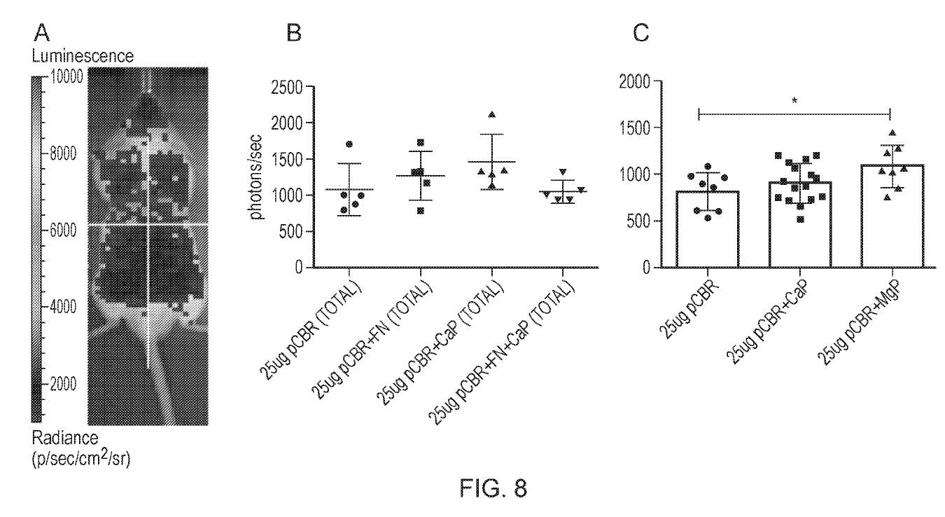

Luciferase imaging 2 weeks post implantation demonstrated luciferase activity for all groups, indicating the potential of agarose to act as a GAM for in vivo gene delivery (FIG. 8 A, B, C). Magnesium-phosphate containing matrices without fibronectin showed a significant enhancement of gene delivery efficacy (FIG. 8C) compared to uncomplexed pCBR pDNA, demonstrating the enhancement of gene delivery in vivo through phosphate salt nanoparticle complexation of the pDNA payloads.

### Example 4: Gene Delivery In Vitro Using Magnesium- and Cobalt-Phosphate Nanoparticles

4.1 Material and Methods

4.1.1. GAM Preparation

GAMs are prepared using the electrophoretic method adapting above-described protocols for in vitro GAMs (see Example 1, section 1.1.1, above) but divalent calcium-cations are replaced by either magnesium or cobalt ions (provided as magnesium-chloride or cobalt-chloride solutions) in the protocol to lead to the formation of either magnesium-phosphate or cobalt-phosphate precipitates nanoparticles using METHOD1 or METHOD2 (preparation with or without fibronectin) or a modified METHOD1 or METHOD2.

4.1.2 In Vitro Transfection

The matrices prepared by METHOD1 and METHOD2 for cell culture are preconditioned with 100 μl of DMEM for 2 hours prior to seeding. Then approximately 5×10⁴ C2C12 cells are seeded onto the scaffolds in a 96-well plate in 15 μl DMEM for 2 hours and subsequently supplemented with 200 μl growth medium (DMEM containing 4.5 g/L glucose, 5% fetal bovine serum, 4 mM L-glutamine and 1% penicillin/streptomycin) and cultured at 37° C., 5% CO₂, humidified atmosphere in the cell culture incubator for up to 4 weeks. Supernatants containing the secreted luciferase reporter gene were sampled at 48 hours, 1 week and 4 weeks post seeding for gene expression monitoring and where possible microscopic images of GFP fluorescent cells are taken.

Metridia luciferase activity is determined using coelenterazine provided as a kit using the manufacturer's instructions (Ready-To-Glow™ protocol, Clontech) and quantified in a Varioskan Flash plate luminometer using white 96-well plates. Metridia luciferase activity is calculated in fold-activity compared to agarose GAM control matrices containing only DNA without calcium phosphate precipitation.

### Example 5: Multi-Gene Delivery In Vitro and In Vivo Using Agarose Gene-Activated Matrices

5.1 Material and Methods

5.1.1 GAM Preparation

GAMs for in vivo implantation are prepared using similar protocols as for in vitro GAMs (see above) but containing an increased amount of pDNA (25 μg). In order to demonstrate multi-gene delivery capabilities in different areas of the constructs, 2 different luciferase plasmids are employed, a red-shifted luciferase to be encoded in the plasmid pCBR and a green-shifted luciferase to be encoded in the plasmid pCBG99. 25 μg of both plasmids are loaded on opposing sides of the matrix, using 2 loading slots at the top and bottom end of the agarose slice using polarity switching and sequential loading. The complexation is carried out at a calcium:phosphate ratio of approximately 166.67-fold of loaded Ca2+ to phosphate buffer for each plasmid, with 60V for 10 min for pDNA1 (pCBR) loading and for 5 min for pDNA2 from the opposing end using reversed polarity. Complexation is carried out at 60V for 5 min for the zone containing pDNA1 and then again using the same parameters but using reversed polarity for complexation in the zone containing pDNA2. GAMs are either prepared without addition of fibronectin (METHOD1) or with the addition of 10 μg of bovine fibronectin during the pDNA loading steps (METHOD2). After complexation DNA/Calcium phosphate bands obtainable by METHOD1 and METHOD2 are excised using a scalpel and individual agarose scaffolds frozen at −86° C. and then lyophilised overnight at 0.0010 millibars (Christ Alpha 2-4 LD_{Plus} lyophiliser). All samples are sterilised by incubation in 70% Ethanol for 24 h and lyophilised again to remove ethanol.

Control samples containing only pDNA1 and pDNA2 without complexation are obtained in the same way but excised directly after the first loading step and lypohilised as described above for complexed samples. Additional controls containing either only pDNA1 (pCBR) or pDNA2 (pCBG99) as imaging controls are prepared according to the protocol above.

5.1.2 In Vitro Evaluation of Dual-Luciferase Activity

Matrices prepared by METHODS and METHOD2 for cell culture are preconditioned with 100 μl of DMEM for 2 hours prior to seeding. Then 5×10⁴ C2C12 cells are seeded onto the scaffolds in a 96-well plate in 15 μl DMEM for 2 hours and subsequently supplemented with 200 μl growth medium (DMEM containing 4.5 g/L glucose, 5% fetal bovine serum, 4 mM L-glutamine and 1% penicillin/streptomycin) and cultured at 37° C., 5% CO₂, humidified atmosphere in the cell culture incubator for up to 4 weeks. Luciferase activity is measured at 7 days, 14 days and 4 weeks, 5 min after addition of 1 mM D-luciferin to the wells in a Xenogen IVIS Spectrum imaging system at 37° C. and individual luciferase signals are obtained by spectral unmixing of distinct wavelengths of CBR and CBG99 luciferase.

5.1.3 In Vivo Implantation

24 h post preparation the matrices are subcutaneously implanted in the backs of male outbred ME-1 mice (5 weeks, 25-30 g, Charles River) under inhalation anaesthesia (Isoflurane 3% for induction, 1.5% for maintenance, 1 L/min O₂) and pockets are closed using resorbable sutures (VICRYL*rapide, polyglactin 910, Ethicon; Johnson & Johnson). 4 samples are implanted per animal (resulting in 4 imaging quadrants) and samples of the 4 groups (only pDNA1+pDNA2, pDNA1+pDNA2+calcium phosphate, pDNA1+pDNA2+calcium phosphate+fibronectin) are applied in a randomised, blocked design. A separate cohort is assigned for the control matrices containing pDNA1+calcium phosphate, pDNA1+calcium phosphate+fibronectin, pDNA2+calcium phosphate, pDNA2+calcium phosphate+fibronectin. Animals receive 0.125 mg/kg buprenorphine (Vetergesic, Alstoe Veterinary) for analgesia intraoperatively as subcutaneous injection. Postoperative antibiosis is administered for 1 week using Baytril® 0.25 mg/ml (Enrofloxacin, Bayer HealthCare Animal Health Division) in the drinking water provided ad libitum.

5.1.4 In Vivo Bioluminescence Imaging

In vivo dual-luciferase imaging is imaged on a Xenogen IVIS Spectrum imaging station 2 weeks post implantation. Animals each receive a 100 μl injection of 5 mg D-luciferin potassium salt (Promega) in physiologic NaCl intraperitoneally prior to imaging and bioluminescence was quantified using the Living Image Software on the imaging station approx. 15 min post injection. Individual luciferase signals are obtained by spectral unmixing of individual luciferase emission peaks for CBR and CBG luciferase respectively.

### Example 6: Delivery of Functional Therapeutic Genes for Bone Formation In Vitro and In Vivo

6.1 Material and Methods

6.1.1 GAM Preparation

GAMs for in vivo implantation in functional assays are prepared using similar protocols as for 3.1.1 but containing an osteoinductive bone morphogenetic protein 2 and 7 (BMP2/7) co-expressing plasmid (25 μg). The matrices are prepared at a calcium:phosphate ratio of 166.67-fold of loaded Ca2+ to phosphate buffer, with 60V for 5 min for pDNA (for the in vivo studies a red-shifted click beetle luciferase, CBR in the plasmid pCBR Control (Promega) is used), loading and 60V for 5 min reversed polarity for complexation. GAMs are either prepared without addition of fibronectin (METHOD1) or with the addition of 10 μg of bovine fibronectin during the pDNA loading step (METHOD2). After complexation DNA/Calcium phosphate bands obtainable by METHOD1 and METHOD2 are excised using a scalpel and individual agarose scaffolds are frozen at −86° C. and then lyophilised overnight at 0.0010 millibars (Christ Alpha 2-4 LD_{Plus} lyophiliser). All samples are sterilised by incubation in 70% Ethanol for 24 h and lyophilised again to remove ethanol.

Control samples containing only pDNA are obtained in the same way but excised directly after the first loading step and lypohilised as described above for complexed samples. Additional controls for the osteoconductive background action of calcium-phosphate itself are prepared without the addition of any pDNA and with or without fibronectin in order to be able to appropriately assess the amount of bone formation induced by the therapeutic BMP2/7 plasmid.

6.1.2 In Vitro Evaluation of Osteogenic Differentiation

24 h post preparation and processing, 5×10⁴ C2C12 cells are seeded onto the scaffolds in a 24-well plate in 200 μl DMEM for 2 hours and subsequently supplemented with 1 ml differentiation assay medium (DMEM containing 4.5 g/L glucose, 1% fetal bovine serum, 4 mM L-glutamine and 1% penicillin/streptomycin) and cultured at 37° C., 5% CO₂, humidified atmosphere in the cell culture incubator for 14 days. On day 14 the matrices are removed and the cell lawn washed once with 1× phosphate buffered saline (PBS) and then washed once with alkaline-phosphatase (ALP) assay buffer. The cells are lysed with 100 μl lysis buffer (ALP-buffer containing 0.25% Triton X-100) on room temperature for 1 h on a plate shaker and then 100 μl of ALP-buffer containing 7.4 mg/ml (20 mM) p-Nitrophenyl phosphate (pNPP) is added and the plate incubated for 20 min in the dark at 37° C. The samples are then transferred to sterile Eppendorf tubes, centrifuged at 13.000 rpm for 2 min and then 100 μl of cleared lysate/reaction mix are measured at 405 nm on a plate reader (Varioskan Flash). The obtained optical densities (OD₄₀₅) and a standard curve are used to calculate the amount of the released ALP-enzyme reaction product p-Nitrophenol per minute, which gives a direct indication of the extent of osteogenic differentiation induced by rhBMP2 in C2012 cells.

6.1.3 In Vivo Implantation

24 h post preparation the matrices are intramuscularly implanted in the gastrocnemius muscle in the hindlimbs of male outbred MF-1 mice (5 weeks, 25-30 g, Charles River) under inhalation anaesthesia (Isoflurane 3% for induction, 1.5% for maintenance, 1 L/min O₂) and pockets are closed using resorbable sutures (VICRYL*rapide, polyglactin 910, Ethicon; Johnson & Johnson). 2 samples are implanted per animal and samples of the investigated groups (pDNA alone, pDNA+calcium phosphate, pDNA+calcium phosphate+fibronectin, only calcium phosphate and calcium-phosphate+fibronectin) are applied in a randomised design. Animals receive 0.125 mg/kg buprenorphine (Vetergesic, Alstoe Veterinary) for analgesia intraoperatively as subcutaneous injection. Postoperative antibiosis is administered for 1 week using Baytril® 0.25 mg/ml (Enrofloxacin, Bayer HealthCare Animal Health Division) in the drinking water provided ad libitum.

6.1.4 μCT Analysis of Bone Formation

4 weeks post-implantation animals are sacrificed using approved Schedule 1 protocols and hindlimb explants are obtained for in vitro pCT analysis using standard protocols. In order to be able to distinguish pre-formed calcium-phosphate precipitates from endogenously formed bone matrix, a separate, in vitro GAM construct is prepared using only calcium-phosphate at the same concentration as in all other samples to be used as an imaging phantom to define suitable grey-value thresholds. Bone volumes and bone mineral densities are quantified and images rendered using Scanco imaging software.

6.1.5 Histological Analysis of Bone Formation

After μCT analysis, explants are additionally investigated using histology to further determine endogenous bone formation using standard protocols. Briefly, ethanol-fixed samples are cut for histological slides and stained for mineralisation using von Kossa staining. A separate set of sections is prepared for immunohistochemistry and stained for osteocalcin in order to define tissue areas with ongoing osteogenic differentiation.

Throughout the description and claims of this specification, the words “comprise” and “contain” and variations of them mean “including but not limited to” and they are not intended to (and do not) exclude other moieties, additives, components, integers or steps. Throughout the description and claims of this specification, the singular encompasses the plural unless the context otherwise requires. In particular, where the indefinite article is used, the specification is to be understood as contemplating plurality as well as singularity, unless the context requires otherwise.

Features, integers, characteristics or groups described in conjunction with a particular aspect, embodiment or example of the invention are to be understood to be applicable to any other aspect, embodiment or example described herein unless incompatible therewith. All of the features disclosed in this specification (including any accompanying claims, abstract and drawings), and/or all of the steps of any method or process so disclosed, may be combined in any combination, except combinations where at least some of the features and/or steps are mutually exclusive. The invention is not restricted to any details of any foregoing embodiments. The invention extends to any novel one, or novel combination, of the features disclosed in this specification (including any accompanying claims, abstract and drawings), or to any novel one, or any novel combination, of the steps of any method or process so disclosed.

The reader's attention is directed to all papers and documents which are filed concurrently with or previous to this specification in connection with this application and which are open to public inspection with this specification, and the contents of all such papers and documents are incorporated herein by reference.

1. A biocompatible material for delivering a biological molecule to target location, the material comprising:
a) a hydrogel matrix material;
b) a divalent cation-phosphate nanoparticle; and
c) a biological molecule, wherein the nanoparticle is encompassed within the hydrogel matrix material.

2. The biocompatible material according to claim 1, wherein the nanoparticle is complexed with the biological molecule, wherein the biological molecule is a biologically active molecule.

3. (canceled)

4. The biocompatible material according to claim 1, wherein the hydrogel matrix material comprises a material selected from the group consisting of hyaluronic acid, polyethylene glycol, agarose, collagen, alginate, chitosan, poly(lactic) acid, poly(lactic-co-glycolic) acid, fibrin, platelet-rich plasma gel and combinations thereof.

5.-8. (canceled)

9. The biocompatible material according to claim 1, wherein the biological molecule is selected from the group consisting of a therapeutic agent or precursor thereof, a nucleic acid molecule, a polypeptide and a cell.

10. The biocompatible material according to claim 9, wherein the nucleic acid molecule is a single stranded nucleic acid molecule or a double stranded nucleic acid molecule, wherein the single stranded nucleic acid molecule is selected from the group consisting of a miRNA, an RNA aptamer and a DNA aptamer, and/or wherein the double-stranded nucleic acid molecule is selected from a gene, siRNA, pDNA, a synthetic gene (linear, 5′ and 3′ end-hairpin ligated expression cassette) and synthetic messenger RNA (mRNA).

11.-13. (canceled)

14. The biocompatible material according to claim 1, wherein the biological molecule is a nucleic acid molecule encoding a polypeptide, or wherein the nucleic acid molecule is a plasmid or vector encoding a plurality of polypeptides.

15. (canceled)

16. The biocompatible material according to claim 1, wherein the polypeptide or plurality of polypeptides is selected from a growth factor, a cytokine, an antibody, an antibody fragment and an extracellular matrix protein, and further wherein, if the polypeptide is a growth factor, it is selected from the group consisting of basic fibroblast growth factor (bFGF, or FGF-2), acid fibroblast growth factor (aFGF), epidermal growth factor (EGF), heparin binding growth factor (HBGF), fibroblast growth factor (FGF), vascular endothelium growth factor (VEGF), transforming growth factor, (e.g. TGF-α, TGF-β, and bone morphogenic proteins such as BMP-2, -3, -4, -6, -7), Wnts, hedgehogs (including sonic, indian and desert hedgehogs), noggin, activins, inhibins, insulin-like growth factor (such as IGF-I and IGF-II), growth and differentiation factors 5, 6, or 7 (GDF 5, 6, 7), leukemia inhibitory factor (LIF/HILDA/DIA), Wnt proteins, platelet-derived growth factors (PDGF), bone sialoprotein (BSP), osteopontin (OPN), CD-RAP/MIA, SDF-1(alpha), HGF and parathyroid hormone related polypeptide (PTHrP).

17-19. (canceled)

20. The biocompatible material according to claim 1, wherein the biological molecule is a cell, and wherein the cell is selected from the group consisting of a neural cell (e.g. a neuron, a oligodendrocytes, a glial cell, an astrocyte), a lung cell, a cell of the eye (e.g. a retinal cell, a retinal pigment epithelial cell, a corneal cell), an epithelial cell, a muscle cell, a bone cell (e.g. a bone marrow stem cell, an osteoblast, an osteoclast or an osteocyte), an endothelial cell, a hepatic cell and a stem cell.

21. The biocompatible material according to claim 1, wherein the divalent cation is selected from Ba²⁺, Co²⁺, Mg²⁺ and Sr²⁺.

22. The biocompatible material according to claim 1, wherein the nanoparticle further comprises a branched or linear amine-containing cationic poly-cation, wherein optionally the branched or linear amine-containing cationic poly-cation is poly-ethylene imine (PEI).

23. (canceled)

24. The biocompatible material according to claim 1, which comprises a plurality of divalent cation-phosphate nanoparticles,
wherein the plurality of divalent cation-phosphate nanoparticles is dispersed within the hydrogel matrix material, and/or
wherein the plurality of divalent cation-phosphate nanoparticles comprises a first set of divalent cation-phosphate nanoparticles having a first predetermined spatial distribution with respect to the hydrogel matrix material and a further set of divalent cation-phosphate nanoparticles having a further pre-determined spatial distribution with respect to the hydrogel matrix material, and/or
wherein the first predetermined spatial distribution differs from the further predetermined spatial distribution, and/or
wherein the first predetermined spatial distribution and/or the further predetermined spatial distribution each create a concentration gradient of the biological molecule and/or nanoparticle distribution.

25.-27. (canceled)

28. The biocompatible material according to claim 1,
wherein the plurality of divalent cation-phosphate nanoparticles comprises a first set of divalent cation-phosphate nanoparticles and a further set of divalent cation-phosphate nanoparticles, wherein the nanoparticles of the first set comprise at least one predetermined characteristic and the nanoparticles of the further set comprise at least one further predetermined Characteristic, and/or
wherein the first set of divalent cation-phosphate nanoparticles differs in at least one characteristic from the further set of divalent cation-phosphate nanoparticles, and/or
wherein the at least one first characteristic and the at least one further characteristic are independently selected from:
a) particle size;
b) type of divalent cation;
c) type of biological molecule;
d) rate of biological molecule release;
e) concentration of biological molecule; and
f) a combination of (a) to (e).

29.-32. (canceled)

33. The biocompatible material according to claim 1, which comprises a bioactive agent wherein the bioactive agent is a polypeptide selected from the group consisting of an extracellular matrix protein e.g. fibronectin, laminin and/or heparin.

34. A three-dimensional scaffold comprising the biocompatible material according to claim 1,
wherein the biocompatible material comprises a plurality of divalent cation-phosphate nanoparticles, wherein the plurality of divalent cation-phosphate nanoparticles comprises a first set of divalent cation-phosphate nanoparticles and a further set of divalent cation-phosphate nanoparticles,
further wherein the nanoparticles of the first comprise at least one predetermined characteristic and the nanoparticles of the further set comprise at least one further predetermined characteristic,
further wherein the scaffold comprises a first zone and a further zone, said first zone comprising a majority of the first set of divalent cation-phosphate nanoparticles and the second zone comprising a majority of the second set of divalent cation-phosphate nanoparticles,
further wherein the first set and the second set differ in at least one predetermined characteristic,
further wherein the first zone a first end of the scaffold and the further zone is a further end of the scaffold,
further wherein the further zone is a second zone and the scaffold further comprises a third zone, and
further wherein the third zone is provided between the first zone and the second zone.

35.-38. (canceled)

39. The three-dimensional scaffold according to claim 1,
wherein the three-dimensional scaffold comprises:
(i) a first set of divalent cation-phosphate nanoparticles which are associated with a biological molecule which is chondrogenic,
wherein the biological molecule is a polypeptide selected from the group consisting of BMP-6, BMP-7, TGF-β3, CD-RAP/MIA and combinations thereof or a nucleic acid encoding a polypeptide selected from BMP-6, BMP-7, TGF-β3, CD-RAP/MIA and combinations thereof, and/or
(ii) first set of divalent cation-phosphate nanoparticles which are associated with a biological molecule which is osteogenic,
wherein the biological molecule is a polypeptide selected from the group consisting of BMP-2 and BMP-7 and combinations thereof, and/or heterodimeric BMP e.g. BMP2/6 or BMP4/7 or a nucleic acid molecule encoding a polypeptide selected from BMP-2 and BMP-7 and combinations thereof and/or heterodimeric BMP e.g. BMP2/6 or BMP4/7.

40.-47. (canceled)

48. A vaccine composition comprising the biocompatible material according to claim 1 or the three-dimensional scaffold according to claim 34, wherein the biological molecule is an immunogenic molecule or an antigen encoding nucleic acid molecule.

49.-50. (canceled)

51. A method of preparing a biocompatible material, the biocompatible material comprising:
a) a hydrogel matrix material;
b) a divalent cation-phosphate nanoparticle; and
c) a biological molecule, wherein the nanoparticle and the biological molecule are encompassed within the hydrogel matrix material,
wherein the method comprises:
i) providing a hydrogel matrix material disposed between a cathode and an anode;
ii) supplying phosphate ions to the hydrogel matrix material;
iii) supplying a solution comprising a biological molecule to the hydrogel matrix material;
iv) supplying a solution comprising a divalent cation to the hydrogel matrix material; and
v) applying an electrical field to the hydrogel matrix material between the cathode and the anode such that a divalent cation-phosphate nanoparticle associated with a biological molecule is formed within the hydrogel matrix material.

52. The method according to claim 51, wherein the phosphate ions are comprised in a buffer solution and step (ii) comprises supplying the buffer solution to the hydrogel matrix material, further wherein the method further comprises step (vi) of supplying a buffer solution to the hydrogel matrix material, and wherein steps (i) to (iv) and (vi) may be performed in any order.

53.-54. (canceled)

55. The method according to claim 51, which comprises:
(i) supplying a plurality of solutions comprising a biological molecule, wherein at least a first solution of the plurality of solutions comprises a biological molecule which is a different biological molecule to a biological molecule comprised in a further solution of the plurality of solutions;
(ii) supplying the first solution comprising a biological molecule to a first target location in the hydrogel matrix material and wherein the method further comprises supplying the further solution comprising a biological molecule to a further target location within the hydrogel matrix material; and
(iii) supplying a plurality of solutions comprising a divalent cation to a first target location in the hydrogel matrix material and wherein the method further comprises supplying the further solution comprising a divalent cation to a further target location within the hydrogel matrix material.

56.-59. (canceled)

60. The method according to claim 51, which comprises:
supplying a plurality of solutions comprising a biological molecule, wherein at least a first solution of the plurality of solutions comprises a biological molecule which is a different biological molecule to a biological molecule comprised in a further solution of the plurality of solutions; and
supplying a plurality of solutions comprising a divalent cation, wherein at least a first solution of the plurality of solutions comprises a divalent cation which is a different divalent cation to a divalent cation comprised in a further solution of the plurality of solutions, wherein each of the plurality of solutions comprising a biological molecule and each of the plurality of solutions comprising a divalent cation are supplied to a common region of the hydrogel matrix material, and further wherein the method further comprises alternating the polarity of the electric field such that each of the divalent cations and each of the biological molecules move to a common target location in the hydrogel matrix material.

61. The method according to claim 52, wherein the buffer solution in the gel and electrophoresis system is a cell and DNA-compatible buffer solution, and wherein the method is carried out under non-denaturing conditions, further optionally wherein the buffer solution is an on-TRIS containing buffer solution, such as HEPES.

62.-65. (canceled)

66. The method according to claim 51, wherein the method further comprises soaking or coating the hydrogel matrix material with an extracellular matrix molecule for example fibronectin and laminin and other RGD-sequence containing peptides to enhance cellular attachment.

67. The method of claim 66, wherein the method further comprises:
(i) lyophilising the hydrogel matrix material to form the biocompatible material;
(ii) drying the hydrogel matrix material under supercritical drying conditions to form the biocompatible material, wherein the biocompatible material is an aerogel; or
(iii) melting the hydrogel matrix material to form an injectable biocompatible material, wherein the biocompatible material forms a hydrogel after implantation.

68.-69. (canceled)
